(19) United States
(12) Patent Application Publication
(10) Pub. No.: US 2003/0083360 A1
(43) Pub. Date: May 1, 2003

(54) Compaction process for manufacture of sodium phenytoin dosage form

(76) Inventors: George Crotts, Kintnersville, PA (US); Mebrahtu G. Fessehaie, Mineola, NY (US); Srinivas Raju Gadiraju, Randolph, NJ (US); John Gawel, Clark, NJ (US); Isaac Ghebre-Sellassie, Morris Plains, NJ (US); Ashlesh K. Sheth, Randolph, NJ (US)

Correspondence Address: Mehdi Ganjeizadeh, Warner-Lambert Company, 2800 Plymouth Road, Ann Arbor, MI, 48105, US

(21) Appl. No.: 10/145,063

(22) Filed: May 14, 2002

Related U.S. Application Data
(60) Provisional application No. 60/290,970, filed on May 15, 2001.

(51) Int. Cl. A61K 9/20; B27N 3/00; A61K 31/4166

(52) U.S. Cl. 514/389; 264/109; 424/465

(57) ABSTRACT

A process for the roller compaction and manufacture of a pharmaceutical formulation comprises the steps of adding sodium phenytoin to a vessel of a blender and adding at least one excipient to the vessel. The mixture is blended and transferred to a roller compactor, where pressure is applied to the blend of sodium phenytoin and excipient. Next, the resultant compaction is milled to form a granulation, which is blended a second time and is suitable for further processing into a dosage form. Preferably, the excipients include magnesium stearate, sugar, lactose monohydrate, and talc. In an alternative embodiment, talc is added immediately prior to the granulation being blended for a second time.

This application claims priority to U.S. Provisional Application No. 60/290,970, the entire contents of which are herein incorporated by reference.

### FIELD OF THE INVENTION

The present invention pertains to a method of manufacturing a dosage form of sodium phenytoin. In particular, the present invention pertains to a method of manufacturing an orally administered extended release sodium phenytoin capsules.

### BACKGROUND OF THE INVENTION

In the pharmaceutical development art, a sustained release dosage form may be defined as a preparation which releases a drug, in vivo, at a considerably slower rate than is the case from an equivalent dose of a conventional (nonsustained release) dosage form. The objective of employing a sustained release product is to obtain a satisfactory drug response while at the same time, reducing the frequency of administration and maintaining bioequivalence to existing sodium phenytoin formulations. An example of a drug, which is popularly used in a sustained release form, is chlorpheniramine maleate. In conventional form, the drug may be given as 4 mg doses every 4 hours or in sustained release form as one dose of 12 mg every 12 hours.

Sustained release compositions for the sequential or timed release of medicaments are well-known in the art. Generally, such compositions contain medicament particles, normally administered in divided doses 2 or 3 times daily, mixed with or covered by a material which is resistant to degradation or disintegration in the stomach and/or in the intestine for a selected period of time. Release of the medicament may occur by leeching, erosion, rupture, diffusion or similar actions depending upon the application of the material. In certain cases, release of hydrophilic material from a formulation can be retarded by application of hydrophobic material.

It is known that different pharmaceutical preparations of the same active ingredient will result in different bioavailabilities of the active ingredient to the mammal. Bioavailability or biological availability may be defined as the percentage of the drug liberated from the dosage form administered that becomes available in the body for biological effect. Different formulations of the same drug can vary in bioavailability to a clinically relevant extent and variation may even occur between batches of the same product due to subtle variations in manufacturing procedures.

Many drugs that are usually administered in tablet or capsule form have a low solubility in biological fluids. For many drugs of low solubility, there is considerable evidence that the dissolution rate partially or completely controls the rate of absorption. Bioavailability can also be affected by a number of factors such as the amounts and types of adjuvants used, the granulation process, compression forces (in tablet manufacturing), the surface area available for dissolution and environmental factors such as churning in the gastrointestinal tract and the presence of food. Due to these numerous factors, specific formulations play an important role in the preparation of prolonged action solid dosage forms. Prolonged action solid dosage forms can be of value in treating diseases such as epilepsy.

Epilepsy is an ancient disease, which affects about 1% of the global population. Despite the progress made in antiepileptic drug therapy, there are still many patients who continue to suffer from uncontrolled seizures and medication toxicity. Examples of major antiepileptic drugs currently in use are: divalproic sodium, ethosuccimide, sodium phenytoin, carbamazepine, and valproic acid.

Pharmacological activity, in general, and antiepileptic activity in particular, correlate better with a concentration of the drug in the blood (or in some other biophase) than with the administered dose. This phenomenon is due, in part, to variability in drug absorption and disposition between and within individuals, particularly when the drug is given orally. Optimizing drug therapy aims at achieving and maintaining therapeutic and safe drug concentrations in the patient's plasma.

Phenytoin, 5,5-diphenyl-2,4-imidazolidinedione, is a well-known pharmaceutical agent having anti-convulsant and antiepileptic activity. Due to phenytoin's poor solubility in water, sodium phenytoin, which is much more soluble, is employed in the preparation of injectable solutions of the drug and in solid dosage forms.

Sodium phenytoin has the following formula:

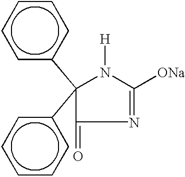

While phenytoin is the antiepileptic drug of choice for many types of epileptic seizures, therapeutic drug monitoring is required because of the difficulty in maintaining an effective therapeutic plasma level of between 10 μg/mL and 20 μg/mL. In addition to the problems of narrow therapeutic plasma levels, phenytoin exhibits great variations in bioavailability following its oral administration to patients because of its poor water solubility.

Even with the new approaches to phenytoin delivery (i.e., Parke-Davis' Dilantin® Kapseals®, which are 100 mg extended sodium phenytoin capsules), it is still necessary for patients to take the drug several times a day to maintain an effective therapeutic plasma level without side effects. With Kapseals®, product in vivo performance is characterized by a slow and extended rate of absorption with peak blood concentrations expected in 4 to 12 hours.

While many techniques and processes have been attempted to provide a reliable dosage form of phenytoin comparable to the Dilantin® Kapseals®, none have been found to be completely satisfactory. Karakasa et al., Biol. Pharm. Bull., 1994;17(3):432-436 in an article entitled “Sustained Release of Phenytoin Following the Oral Administration of Sodium Phenytoin/Ethylcellulose Microcapsules in Human Subjects and Rabbits,” studied the release patterns of phenytoin as the sodium salt in combination with ethylcellulose. The sodium phenytoin microcapsules were prepared by mixing 80% (by weight) of the sodium phenytoin in a 10% (by weight) ethylcellulose solution in ethyl acetate. The suspension was stirred and n-pentane was added dropwise until a phase separation occurred and the microcapsules were obtained. The microcapsules were collected on filter paper, dried and stored. Karakasa et al. point out that following the oral administration of sodium phenytoin, the salt might be easily transferred into free-phenytoin in the acidic fluids of the stomach. As free-phenytoin is practically insoluble in water, its absorption might be incomplete in the gastrointestinal tract. On the other hand, while passing through the stomach, the volume of water penetrating into the ethylcellulose microcapsules might be minimal. Thus, most of the sodium phenytoin in the microcapsules might not be converted into free-phenytoin.

A review article by Boxenbaum in Drug Development & Industrial Pharmacy, 1982;8(v): 1-25, entitled “Physiological and Phamacokinetic Factors Affecting Performance of Sustained Release Dosage Forms” actually suggests that sustained release formulations for drugs such as phenytoin are unnecessary. Boxenbaum points out that dosing schedules of once a day versus 3 times daily produce similar plasma curves. This results from both the slow absorption and the low solubility of the drug.

Slow release, delayed release, prolonged release, or sustained release phenytoin is a desirable objective. Controlled release oral dosage forms of drugs with long half-lives, such as phenytoin, have been disregarded for sustained release formulation since they produce little change in the blood concentration after multiple doses have been administered. The existence of such products can, however, be justified, on the basis of their ability to minimize toxicity and the occurrence of adverse reactions and as providing greater patient convenience and thus, better patient compliance.

A paper by Bourgeois entitled “Important Pharmacokinetic Properties of Antiepileptic Drugs” in Epilepsia, 1995;36(Supp. 5), discusses the important pharmacokinetic properties of antiepileptic drugs. The author states that a drug's rate of absorption profile is described by its absorption constant (k_{abs}). A high absorption constant results in early and high peak serum concentrations. A high (k_{abs}) value also results in greater fluctuations in drug levels compared with the steadier concentrations resulting from lower (k_{abs}) values. A lower absorption constant can often be produced by formulating an otherwise rapidly absorbed drug in a slow release preparation. However, using enteric coated preparations as part of the process of manufacturing a dosage form does not alter a drug's (k_{abs}) value, they merely delay absorption. An enteric coating is designed to prevent absorption in the acidic environment of the stomach. Consider for example, a patient who has received a single dose of enteric-coated valproate. For the first few hours after dosing, serum measurements will fail to detect any drug in the blood. Not until the tablet reaches the alkaline environment of the duodenum does the serum concentration rapidly increase, ultimately achieving a profile similar to that of an uncoated preparation of valproate. Therefore, the enteric coating merely shifts the time concentration profile to the right.

From a review of the prior art, it is evident that a need still remains for a process that can readily and consistently produce a sustained release dosage form for drugs with pH dependent solubilities, such as sodium phenytoin, which provides initial therapeutic levels of the drug and delays the delivery of another fraction of the drug to eliminate excess concentrations for about 1 to 5 hours. The processes of the invention are useful for producing a dosage form of sodium phenytoin that has a substantially consistent dissolution profile.

### SUMMARY OF THE INVENTION

The present invention meets the unfulfilled needs described above by providing a process for readily producing a formulation that has a given proportion of a required dose. When sodium phenytoin is the active pharmaceutical ingredient, the formulation exhibits bioequivalency to Dilantin® Kapseals® dosage forms. Specifically, the present invention comprises the use of a roller compaction process to form consistent granules, which upon encapsulation provide a predictable dissolution profile. More specifically, the present invention comprises the use of a roller compaction process to form consistent granules which upon encapsulation provide a substantially consistent dissolution profile among various lots of dosage formulation blends comprising the same bulk substance sodium phenytoin. The process also produces a reliable and consistent product of sodium phenytoin. Therefore, standard application of this process provides a reliable manufacturing process of sodium phenytoin dosage forms as well as assuring consistent product performance.

In general, the present invention provides a process for the manufacture of a pharmaceutical product. The process comprises the steps of adding sodium phenytoin to a vessel or bowl of a blender and adding at least one excipient to the vessel. Next, the mixture is blended to form a blend. The resultant blend is transferred to a roller compactor and compacted between at least two rollers to form a compact with the excipient. The pressure imparted on the blend enhances the physical adhesion between the sodium phenytoin and the excipient. The compact is subsequently milled to form a granulation. The resultant granulation is then formed into the desired dosage form, such as capsules.

In one embodiment of the invention, the process comprises the steps of adding sodium phenytoin to a vessel of a blender; adding an excipient to the vessel; blending the sodium phenytoin and the excipient to form a first blend; compacting the first blend with sufficient force between at least two rollers to cause a portion of the sodium phenytoin to fracture and form a compact, wherein the rollers apply a force of between 1 and 20 kilo-Newtons (kN) to the first blend, the rollers rotate at a speed of between 1 and 20 rpm, and wherein the outer edge of said rollers are positioned between 0.5 mm and 5 mm apart at their closest point; milling the compact to form a granulation; and blending the granulation to form a second blend.

Another embodiment of the invention, the rollers apply a force of 2.5 kN, the rollers rotate at a speed of 10 rpm, and the outer edge of the rollers are positioned 3 mm apart at their closest point.

In another embodiment of the invention, the excipients include magnesium stearate, sugar and lactose monohydrate and the process includes the step of blending talc with the sodium phenytoin granulation. Alternatively, the talc may be included as one of the excipients initially mixed with the sodium phenytoin in the vessel.

Further, patients will benefit from such a formulation since many drugs, like sodium phenytoin, have narrow therapeutic windows, which could require multiple (three or more) daily dosings.

It is to be understood that both the foregoing general description and the following detailed description are exemplary, but are not restrictive, of the invention.

The invention is best understood from the following detailed description when read in connection with the accompanying drawing, in which:

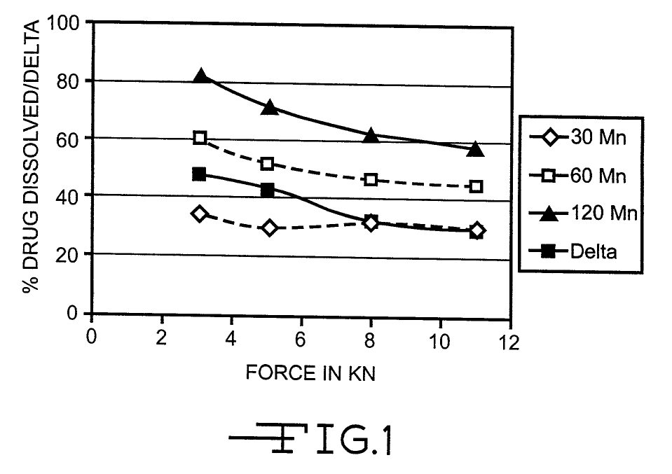

FIG. 1 is a graphical representation showing dissolution of a mixture manufactured by the process of the present invention at various compaction forces using same roller gap and speed;

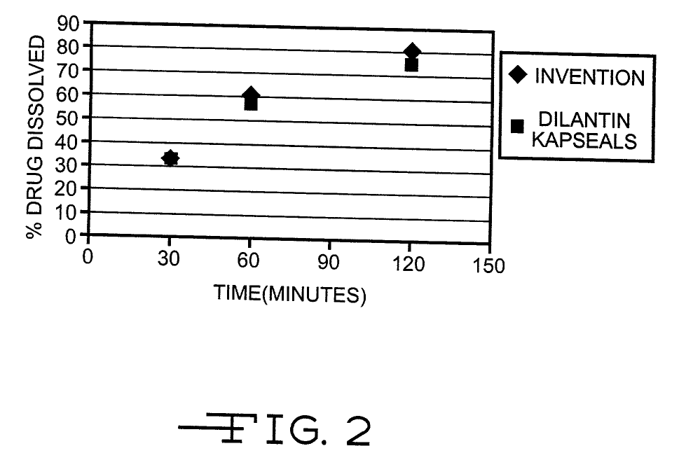

FIG. 2 is a graphical representation showing the dissolution profile of two sodium phenytoin formulations produced by the process of the present invention compared to the dissolution profile of a Dilantin® Kapseals® dosage form.

### DETAILED DESCRIPTION OF THE INVENTION

The present invention comprises a roller compaction process, which is applied to a mixture of an active pharmaceutical ingredient and one or more excipients to form granules with consistent characteristics. In particular, the present invention is a process for the production of granules of the active pharmaceutical ingredient sodium phenytoin.

The process of the present invention involves the use of a roller compaction device having variable rotation speed, force application, and gap width capabilities. A Gerteis Polygran dry roller compactor system having 100-mm knurled rollers, commercially available from Gerteis of Germany, is a preferred roller compaction device because the programmable logic control systems of that roller compactor are relatively easy to operate.

The roller compactor functions by uniformly applying pressure on a mixed powder blend by passing the blend between two counter-rotating rollers. The pressure imparted on the blend by the rollers compresses the powder into a compact, such as a sheet or ribbon, which is typically milled to produce granules.

The process of the present invention relates to the discovery that some therapeutic agents, such as sodium phenytoin, can be formulated and processed to yield a dosage form providing sustained blood plasma concentrations of the active pharmaceutical ingredient. It will be understood by the skilled artisan that the effective amounts are released over an intended delivery time and for a desired blood plasma concentration.

It has been found that the controlled application of pressure to a mixture of an active pharmaceutical ingredient and at least one excipient during roller compaction yields a product that is relatively easy to produce yet exhibits sustained release properties in a reproducible manner. Further, in the case of sodium phenytoin, the product is bioequivalent to commercial Dilantin® Kapseals®. More specifically, it is believed that by roller compacting a blend prepared in accordance with the current invention, the ingredients are forced into a state of intimate contact, mixing and adhesion. The particles undergo rearrangement, and it is believed that particle fracturing creates multiple surface sites, contact points and bonding sites between the active pharmaceutical ingredient and the excipient. The enhanced contact between the active pharmaceutical ingredient and excipient directly affects the dissolution properties of the active pharmaceutical ingredient. In other words, it is believed that one or more of the excipients form a drug dissolution inhibiting coating around the active pharmaceutical ingredient upon exposure to the pressure imparted by the roller compactor. This approach provides the means to develop a reproducible process for the manufacture of sodium phenytoin dosage forms.

More specifically, the present invention comprises the use of a roller compaction process to form consistent granules which upon encapsulation provide a substantially consistent dissolution profile among various lots of dosage formulation blends comprising the same bulk substance sodium phenytoin.

By “substantially consistent” dissolution profile is intended to mean that the difference in the percentage dissolution of any two formulation batches of the same bulk substance sodium phenytoin is no greater than 15% when measured under the same conditions (e.g., temperature and time) by well-known methods in the art including those exemplified herein. More preferably, this difference is between 10% and 15%, even more preferably between 5 to 10%, yet even more preferably between 2% and 5%; most preferably between 0% and 2%.

To achieve the objective of the present invention, an active pharmaceutical ingredient is deposited in a vessel of a blender, such as a Patterson-Kelley® twin shell blender. Preferably, sodium phenytoin is the active pharmaceutical ingredient. Unless otherwise indicated herein, the percentages of the constituents shall mean weight percentages. Typically, the active pharmaceutical ingredient is present at about 25% to 75% of the overall weight of the final dose form. Preferably, 35% to 50% is added to the vessel.

Next, excipients such as fillers and lubricants are deposited in the vessel of the blender with the active pharmaceutical ingredient, although the order of addition is not important and may be reversed. Multiple lubricants may be added to the mixture and are well-known in the art, such as stearic acid and magnesium stearate. The lubricant may be added in amounts of about 1% to about 10% of the overall weight of the mixture, preferably 2% to 5%.

The present invention may also contain at least one filler as an excipient. Suitable fillers are well-known in the art and typically comprise microcrystalline cellulose, sorbitol, mannitol, confectioner's sugar, compressible sugar, glucose, lactose monohydrate, and talc. Preferably, confectioner's sugar, lactose monohydrate, compressible sugar, or combinations thereof is added to about 25% to 75% of the overall weight of the mixture. Talc may be added to about 0.5% to 5% of the overall weight of the mixture. Although talc may be added to the vessel of the blender with the other fillers, talc may alternatively be added to the mixture just prior to an additional blending step, as described below. Preferably, one or more of the ingredients are first deplumed before being added to the vessel, such as by passing the ingredients through a screen. Where the blender utilized in the processes of the invention is a twin shell blender, this blender optionally comprises an intensifier bar. By “intensifier bar” is intended a bar containing blades that rotate in a direction opposite to that of the twin shell. Utilization of such bars to improve agitation in the powder bed is well-known in the art.

After all ingredients are added to the vessel, the blender is activated and the mixture is blended in the vessel of the blender. One such blender, described above, which may be used in the present invention is a Patterson-Kelley® blender. The powder mixture is deposited in the blender and blended for about 10 to 60 minutes at a speed of about 5 to 30 rpm.

The resultant blend is subsequently transferred to a roller compactor in a known manner. The roller speed, roller gap width, and force of compaction are then adjusted and the blend is fed through the roller compactor in a known manner. Specifically, the process of the present invention compresses the blend of sodium phenytoin and excipients into compacts by applying an optimal force to form the compact. The preferred force and other conditions can be selected to provide sufficient adhesion among constituents to permit a suitable dissolution profile. One skilled in the art can identify the factors empirically. With respect to a Gerteis roller compactor, the optimal force is typically between 1 and 20 kN. In such a compactor, the optimal force is preferably between 2 and 6 kN, even more preferably 2.5 kN.

To maintain a steady output of material from the roller compactor, the rollers rotate at a speed of between 1 and 20 rpm. Preferably, the rollers rotate at a speed of between 5 and 15 rpm. Additionally, the outer edge of the rollers are positioned between 0.5 mm and 5 mm apart, with the outer edges of the rollers are preferably positioned between 2 mm and 4 mm apart at their closest point. Although variances in roller rotation speed and roller gap width affect the dissolution profile of the sodium phenytoin, the roller force is the most significant parameter, as described above and detailed in Example 3.

Upon contact with the counter-rotating rollers of the roller compactor, the compression force imparted on the blend by rollers converts the powdered form of the blend into a ribbon or compaction sheet. This compact is subsequently fed to a mill, typically an oscillating mill, fitted with a screen. Preferably, the screen has a hole diameter between 0.2 mm and 2 mm, most preferably about 1.0 mm. After passing through the mill and screen, the compact is converted into a granulation.

After milling, the granulation is transferred to a blender and blended in a similar manner as described above to form a second blend. However, if talc was not added prior to compaction with the other excipients, it may optionally be added prior to this second blending step. Once blended for a second time, the resultant blend may be encapsulated in a known manner such as by using a Höfliger and Karg encapsulation machine. Granules may be filled into the body of the capsule dosage form by tamping or dosing and the capsule may be subsequently sealed using a cap.

As shown in FIG. 1, the compaction force plays a major role on the dissolution of sodium phenytoin. Specifically, it was found that the greater the amount of force applied to the blend fed to the roller compactor, the lower the dissolution rate at constant speed and gap. Thus, adjusting the pressure applied to a blend of active ingredient and excipient fed to the roller compactor can reproducibly control the dissolution profile of sodium phenytoin in a dosage form. Additionally, as shown in FIG. 2, the dosage form prepared in a manner according to the present invention has a similar release profile when compared to Dilantin® Kapseals® dosage forms.

### EXAMPLE 1

A blend of sodium phenytoin and excipients was provided in the amounts described in Table 1. The mixture was blended for 10 minutes in a Patterson-Kelly®.

TABLE 1
Blend Formulation

| Ingredients | % of Overall Weight |
|---|---|
| Sodium Phenytoin, USP | 43.5% |
| Magnesium Stearate, NF | 3.9% |
| Compressible Sugar, NF | 24.9% |
| Talc, USP | 2.7% |
| Lactose Monohydrate, NF | 25.0% |

### EXAMPLE 2

To determine the extent to which the force of compaction plays on dissolution of granules produced by the process of the present invention, the roller gap and roller speed process parameters were held constant, as detailed below. Table 2 provides the dissolution data of a portion of the blend described in Example 1 compressed at varying roller forces. The percent of drug dissolved was determined using standard protocols well-known in the art. Specifically, a USP dissolution test was used for each of the sodium phenytoin formulations. Specifically, this test involves placing each capsule in 900 mL of water, which was maintained at 37° C.±0.5° C. and stirred at 50 rpm. Samples were collected at 30, 60, and 120 minutes and tested for the amount of sodium phenytoin dissolved.

TABLE 2
Effect of Force of Compaction on Dissolution

| Process parameters: | | | |
|---|---|---|---|
| Roller gap = 2 mm | Dissolution (%) | | |
| Roller speed = 3 rpm | (sd) | | |
| Roller Force | n = 12 | | |
| (kilo-Newtons) | 30 Min | 60 Min | 120 Min |
| 5 kN | 32(1.5) | 55(3.4) | 74(3.7) |
| 8 kN | 29(1.4) | 46(2.1) | 62(3.6) |
| 11 kN | 31(2.2) | 46(3.1) | 61(4.4) |
| 14 kN | 29(2.9) | 43(4.1) | 57(5.4) |
| 17 kN | 32(2.4) | 47(3.0) | 62(3.4) |

The data provided in Table 2 indicates that as roller force increases, up until at least 14 kN, the amount of sodium phenytoin that dissolves by 120 minutes decreases.

### EXAMPLE 3

To determine the extent to which the force of compaction alone affects dissolution, all of the process parameters were held constant except the roller force, as detailed above in Table 2. However, Table 3 provides the dissolution data of various samples of the blend described in Example 1 at varying roller forces, roller gap widths (the distance between the outer edge of the rollers at their closest point), and roller speeds. Similar to Example 2, the percent of drug dissolved was determined using standard protocols well-known in the art.

TABLE 3
Effect of Process Parameters

| Process Parameters | | | | Dissolution (%) | | |
|---|---|---|---|---|---|---|
| | Roller | Roller | Roller | (sd) | | |
| Batch | Gap | Speed | Force | n = 12 | | |
| Run No. | (MM) | (RPM) | (KN) | 30 Min | 60 Min | 120 Min |
| 1 | 2.5 | 6.0 | 7.0 | 29(2.0) | 49(3.2) | 66(4.4) |
| 2 | 2.0 | 3.0 | 3.0 | 33(2.9) | 62(57) | 81(4.7) |
| 3 | 2.5 | 6.0 | 11.0 | 27(2.3) | 43(2.6) | 59(3.8) |
| 4 | 3.0 | 3.0 | 11.0 | 27(1.9) | 44(2.3) | 60(3.8) |
| 5 | 2.0 | 6.0 | 11.0 | 28(1.2) | 44(2.9) | 59(4.4) |
| 6 | 2.0 | 6.0 | 11.0 | 29(2.1) | 45(2.8) | 60(3.8) |
| 7 | 2.5 | 6.0 | 7.0 | 28(1.9) | 46(5.1) | 65(6.4) |
| 8 | 3.0 | 9.0 | 11.0 | 27(2.1) | 43(2.7) | 60(4.0) |

It can be seen in Table 3 that the roller force clearly plays the predominant role in determining dissolution profile of the drug product produced in this invention. For example, a comparison of the dissolution data from runs 1, 3, and 7 confirm that an increase in roller force reduces the dissolution rate. On the other hand, statistical analysis reveals that the roller gap width and speed do not affect the dissolution rate to the same extent.

### EXAMPLE 4

The process parameters of the invention were further tested using various preparations of the bulk substance sodium phenytoin. Unless otherwise indicated, all procedures and parameters were according to those described above. The ingredients and the weight ratios shown in Table 1 was kept the same, with the optional substitution of confectioner's sugar for compressible sugar. This data is summarized below in Tables 4-10 and shows that substantially consistent dissolution profiles are achieved for a given sodium phenytoin bulk drug substance.

Three sodium phenytoin bulk drug substances (I, II, and III) were evaluated with the present invention. For bulk drug substance sodium phenytoin I, 80% of the particles were typically between 3-126 microns; with the median (50^{th} percentile) particle size about 15-23 microns (assessed by Coulter counting). The dissolution profiles for bulk drug substance sodium phenytoin I are depicted in Tables 2, 4, 5, 6, 8, and 9.

For bulk drug substance sodium phenytoin II, 45-70% of the particles were typically greater than or equal to 179 microns and 5-30% of the particles were greater than or equal to 44 microns (assessed by sieve analysis). The dissolution profiles for bulk drug substance sodium phenytoin I are depicted in Table 10.

Bulk drug substance sodium phenytoin III appeared to predominantly have a very fine particle size; with the median estimated to be less than 15 microns.

TABLE 4
Dissolution Profiles of Sodium Phenytoin Capsules Using Bulk Drug
Substance Sodium Phenytoin I

| | Process | | | | | |
|---|---|---|---|---|---|---|
| | Parameters: | | | | | |
| | Force (kN), | Na | % of | % Dissolved (SD) | | |
| Batch | Speed (RPM), | Phenytoin | Each Lot | 30 | 60 | 120 |
| No. | Gap (mm) | Lots Used | Used | Min. | Min. | Min. |
| A | 3.2 kN, 7.0 rpm, | 1 | 91.8 | 31 | 52 | 71 |
| | 2.6 mm | 2 | 8.2 | (0.8) | (1.7) | (2.6) |
| B | 3.4 kN, 6.5 rpm, | 2 | 100 | 25 | 45 | 65 |
| | 2.4 mm | | | (1.4) | (2.2) | (1.8) |
| C | 3.0 kN, 7.5 rpm, | 3 | 70.8 | 28 | 49 | 69 |
| | 2.8 mm | 4 | 29.2 | (1.4) | (2.2) | (3.3) |
| D | 3.2 k.N, 7.0 rpm, | 5 | 48.5 | 29 | 49 | 70 |
| | 2.6 mm | 6 | 51.5 | (2.8) | (3.5) | (3.2) |
| E | 3.3 kN, 6.8 rpm, | 5 | 48.5 | 27 | 46 | 67 |
| | 2.5 mm | 6 | 51.5 | (2.2) | (3.3) | (3.2) |
| F | 3.1 kN, 7.3 rpm, | 7 | 48.5 | 30 | 50 | 70 |
| | 2.7 mm | 8 | 51.5 | (1.5) | (2.4) | (3.2) |

Range of process parameters:

Force 3.0 to 3.4 kN

Roller Speed 6.5 to 7.5 rpm

Roller Gap 2.4 to 2.8 mm

TABLE 5
Batches Made at Full Scale (900 kg) to
Demonstrate Process Reproducibility

| Roller Compaction | % Dissolved | | |
|---|---|---|---|
| Batch No. | 30 Min. | 60 Min. | 120 Min. |
| G1 | 30 | 52 | 73 |
| H1 | 30 | 52 | 73 |
| I1 | 31 | 54 | 75 |
| J1 | 32 | 55 | 75 |
| K1 | 34 | 59 | 78 |
| L1 | 34 | 62 | 81 |
| M1 | 35 | 61 | 82 |
| N1 | 38 | 63 | 82 |
| O1 | 35 | 58 | 78 |
| P1 | 31 | 53 | 74 |
| Q1 | 31 | 54 | 75 |
| (Batch A, Table 4) | 31 | 52 | 71 |
| (Batch D, Table 4) | 29 | 49 | 70 |
| Mean | 32 | 56 | 76 |
| SE | 0.72 | 1.2 | 1.1 |
| Median | 31 | 54 | 75 |
| Mode | 31 | 52 | 75 |
| SD | 2.6 | 4.4 | 4.0 |

Parameters: Force = 3.2 kN, Speed = 7.0 rpm, Gap = 2.6 mm

TABLE 6
Process Parameter Optimization Using the Gerteis Roller Compactor

| Roller | | | | |
|---|---|---|---|---|
| Com- | | | | |
| paction | | % Drug Dissolved (SD) | | |
| Batch No. | Process Parameter Description | 30 Min. | 60 Min. | 120 Min. |
| Q1 | Force = 2.0 kN | 36(2.6) | 61(2.9) | 82(2.3) |
| | Constant speed and gap | | | |
| Q2 | Force = 2.5 kN* | 33(2.0) | 58(3.3) | 80(2.5) |
| | Constant speed and gap | | | |
| Q3 | Force = 3.0 kN | 33(1.5) | 56(2.8) | 76(2.1) |
| | Constant speed and gap | | | |
| Q4 | Gap = 2.5 mm | 34(1.5) | 56(2.5) | 76(2.3) |
| | Constant speed and force | | | |
| Q5 | Force = 2.5 kN* | 33(2.0) | 57(3.4) | 77(2.8) |
| | Constant speed and gap | | | |
| Q6 | Gap = 3.5 mm | 33(1.2) | 56(3.0) | 76(3.1) |
| | Constant speed and force | | | |

Constant speed = 10 rpm; Constant force = 2.5 kN; and Constant gap = 3.0 mm.
*Parameters replicated

TABLE 7
Dissolution Data for Various Batches Manufactured Using Bulk
Drug Substance Sodium Phenytoin III

| Roller | | | | |
|---|---|---|---|---|
| Com- | | % Drug Dissolved | | |
| paction | | (SD) | | |
| Batch | | | | 120 |
| No. | Parameters | 30 Min. | 60 Min. | Min. |
| R1 | Force = 6.0 kN, Gap = 2.0 mm, | 38 | 71 | 86 |
| | Speed = 8.0 rpm | (3.2) | (2.7) | (1.2) |
| S1 | Force = 10 kN, Gap = 2.0 mm, | 31 | 58 | 78 |
| | Speed = 8.0 rpm | (2.9) | (2.9) | (1.8) |
| R2 | Force = 6.0 kN, Gap = 2.0 mm, | 40 | 74 | 88 |
| | Speed = 12.0 rpm | (4.3) | (5.5) | (2.7) |
| S2 | Force = 10.0 kN, Gap = 2.0 mm, | 34 | 65 | 86 |
| | Speed = 12.0 rpm | (2.2) | (2.5) | (2.1) |
| R3 | Force = 6.0 kN, Gap = 4.0 mm, | 45 | 71 | 87 |
| | Speed = 8.0 rpm | (3.9) | (3.9) | (3.1) |
| S3 | Force = 10.0 kN, Gap = 4.0 mm, | 32 | 61 | 80 |
| | Speed = 8.0 rpm | (4.0) | (4.5) | (3.1) |
| U1 | Force = 6.0 kN, Gap = 4.0 mm, | 39 | 78 | 90 |
| | Speed = 12.0 rpm | (4.0) | (2.3) | (0.6) |
| U2 | Force = 10.0 kN, Gap = 4.0 mm, | 35 | 69 | 87 |
| | Speed = 12.0 rpm | (2.1) | (3.9) | (2.1) |
| S4 | Force = 8.0 kN* | 34 | 65 | 85 |
| | Gap = 3.0 mm, | (2.9) | (3.1) | (2.6) |
| | Speed = 10.0 rpm | | | |
| R4 | Force = 8.0 kN,* | 40 | 68 | 85 |
| | Gap = 3.0 mm, | (2.6) | (2.3) | (2.1) |
| | Speed = 10.0 rpm | | | |
| U3 | Force = 8.0 kN,* | 37 | 71 | 87 |
| | Gap = 3.0 mm, | (1.7) | (1.4) | (1.4) |
| | Speed = 10.0 rpm | | | |

*Replicated Center Points

TABLE 8
Dissolution Data for Various Batches Manufactured Using Bulk
Drug Substance Sodium Phenytoin I

| Roller | | | | |
|---|---|---|---|---|
| Com- | | % Drug Dissolved | | |
| paction | | (SD) | | |
| Batch | | | | 120 |
| No. | Parameters | 30 Min. | 60 Min. | Min. |
| V1 | Force = 2.0 kN, Gap = 2.5 mm, | 34 | 58 | 76 |
| | Speed = 8.0 rpm | (1.3) | (1.0) | (0.8) |
| V2 | Force = 3.0 kN, Gap = 2.5 mm, | 32 | 56 | 75 |
| | Speed = 8.0 rpm | (1.9) | (2.1) | (1.4) |
| V3 | Force = 2.0 kN, Gap = 2.5 mm, | 32 | 56 | 75 |
| | Speed = 12.0 rpm | (1.0) | (1.6) | (2.0) |
| W1 | Force = 3.0 kN, Gap = 2.5 mm, | 34 | 56 | 75 |
| | Speed = 12.0 rpm | (2.3) | (2.2) | (2.4) |
| W2 | Force = 2.0 kN, Gap = 3.5 mm, | 34 | 57 | 74 |
| | Speed = 8.0 rpm | (3.2) | (4.2) | (2.8) |
| W3 | Force = 3.0 kN, Gap = 3.5 mm, | 32 | 56 | 75 |
| | Speed = 8.0 rpm | (2.1) | (2.3) | (1.7) |
| X1 | Force = 2.0 kN, Gap = 3.5 mm, | 33 | 58 | 76 |
| | Speed = 12.0 rpm | (2.5) | (1.9) | (1.4) |
| X2 | Force = 3.0 kN, Gap = 3.5 mm, | 33 | 56 | 75 |
| | Speed = 12.0 rpm | (0.8) | (1.2) | (2.3) |
| X3 | Force = 2.5 kN, Gap = 3.0 mm,* | 34 | 56 | 74 |
| | Speed = 10.0 rpm | (3.1) | (3.8) | (2.8) |
| W4 | Force = 2.5 kN, Gap = 3.0 mm,* | 32 | 53 | 72 |
| | Speed = 10.0 rpm | (0.8) | (1.0) | (1.6) |
| V4 | Force = 2.5 kN, Gap = 3.0 mm,* | 32 | 56 | 75 |
| | Speed = 10.0 rpm | (1.7) | (1.1) | (0.8) |

*Replicated Center Points

TABLE 9
Process Parameter Optimization at Pilot Scale
(40 kg)

| Na Phenytoin | | | |
|---|---|---|---|
| Lot No.- | | | |
| Roller Compaction | % Drug Dissolved (SD) | | |
| Batch No. | 30 Min. | 60 Min. | 120 Min. |
| I-a | 33(1.9) | 57(2.7) | 77(2.1) |
| I-b | 34(1.1) | 59(1.9) | 78(2.3) |
| II-c | 35(3.1) | 60(2.6) | 79(2.3) |
| III-d | 34(1.5) | 59(2.3) | 78(1.9) |
| IV-e | 32(1.2) | 57(2.4) | 77(2.6) |

Force = 2.5 kN; Gap = 3.0 mm, Speed = 10.0 rpm

TABLE 10
Process Parameter Optimization Using Bulk Drug Substance Sodium
Phenytoin II

| Roller | | | | |
|---|---|---|---|---|
| Com- | | | | |
| paction | | % Drug Dissolved (SD) | | |
| Batch | | | | 120 |
| No. | Process Parameter Description | 30 Min. | 60 Min. | Min. |
| X-1 | F = 10 kN, G = 3 mm, S = 12 rpm | 27(1.3) | 44(1.2) | 61(1.5) |
| X-2 | F = 8 kN, G = 4 mm, S = 4 rpm | 27(1.2) | 46(1.8) | 65(1.0) |
| X-3 | F = 12 kN, G = 2 mm, S = 8 rpm | 25(1.5) | 41(2.1) | 58(2.3) |
| X-4 | F = 6 kN, G = 2.5 mm, | 28(1.8) | 46(2.6) | 65(2.3) |
| | S = 10 rpm | | | |
| Y-1 | F = 2.5 kN, G = 3 mm, | 26(1.3) | 43(2.1) | 62(2.4) |
| | S = 12 rpm | | | |

The data depicted above indicates that various batches of sodium phenytoin formulations made according to the processes of the invention and from the same bulk substance sodium phenytoin demonstrate a substantially consistent dissolution profile.

Although illustrated and described herein with reference to certain specific embodiments and examples, the present invention is nevertheless not intended to be limited to the details shown. Rather, the claims should be read to include various modifications within the scope and range of equivalents of the claims, without departing from the spirit of the invention.

What is claimed is:

1. A process for manufacturing a pharmaceutical formulation comprising the steps of:
adding sodium phenytoin to a vessel of a blender;
adding at least one excipient to said vessel;
blending said excipient and said sodium phenytoin to form a blend;
compacting said blend to form a compact; and
milling said compact to form a granulation.

2. The process according to claim 1, wherein said sodium phenytoin is added to said vessel in an amount of 15% to 45% of the total weight of said granulation.

3. The process according to claim 1, wherein said at least one excipient is selected from the group consisting of at least one of stearic acid, magnesium stearate, microcrystalline cellulose, sorbitol, mannitol, confectioner's sugar, compressible sugar, glucose, lactose monohydrate, and talc.

4. The process according to claim 1, wherein said at least one excipient comprises magnesium stearate.

5. The process according to claim 1, wherein said at least one excipient comprises magnesium stearate, sugar, lactose monohydrate, and talc.

6. The process according to claim 5, wherein said magnesium stearate, sugar, lactose monohydrate, and talc are added to about 25% to 75% of the total weight of said granulation.

7. The process according to claim 5, wherein said magnesium stearate is added from 0.5% to 5% of the total weight of said granulation.

8. The process according to claim 5, wherein said sugars are added from 25% to 75% of the total weight of said granulation.

9. The process according to claim 5, wherein talc is added in an amount of 0.5% to 5% of the total weight of said granulation.

10. The process according to claim 1, wherein said sodium phenytoin is added to 35% to 55% of the total weight of said granulation.

11. The process according to claim 1, wherein the step of compacting comprises compacting said sodium phenytoin and said at least one excipient with a roller compactor having at least two rollers.

12. The process according to claim 11, wherein the step of compacting comprises compacting said sodium phenytoin and said at least one excipient with a force of between 1 and 20 kilo-Newtons between said rollers.

13. The process according to claim 11, wherein the step of compacting comprises compacting said sodium phenytoin and said at least one excipient with a force of between 2 and 5 kilo-Newtons between said rollers.

14. The process according to claim 11, wherein said rollers are rotated at a speed of between 1 and 20 revolutions per minute.

15. The process according to claim 11, wherein said rollers are rotated at a speed of between 5 and 12 revolutions per minute.

16. The process according to claim 11, wherein the outer edge of said rollers are positioned between 1 mm and 5 mm apart.

17. The process according to claim 11, wherein the outer edge of said rollers are positioned between 2 mm and 4 mm apart.

18. The process according to claim 1 wherein said at least one excipient comprises magnesium stearate, sugar, and lactose monohydrate and said process further comprises blending said granulation with talc to form a blend.

19. The process according to claim 18 further comprising the step of forming said blend into a dosage form by encapsulating a portion of said blend.

20. A process for the dry granulation and manufacture of a pharmaceutical formulation, the method comprising the steps of:
adding sodium phenytoin to a vessel of a blender;
adding an excipient to said vessel, wherein said excipient is selected from the group consisting of at least one of stearic acid, magnesium stearate, microcrystalline cellulose, sorbitol, mannitol, sugar, confectioner's sugar, compressible sugar, glucose, and lactose monohydrate;
blending said sodium phenytoin and said excipient to form a first blend;
compacting said first blend to form a compact;
milling said compact to form a granulation;
adding talc to said granulation; and
blending said granulation to form a second blend.

21. The process according to claim 20, wherein said sodium phenytoin is added to said vessel in an amount of 25% to 75% of the total weight of said blend.

22. The process according to claim 20, wherein said excipient comprises magnesium stearate, sugar, compressible sugar, and lactose monohydrate.

23. The process according to claim 22, wherein said magnesium stearate is added from 0.5% to 5% of the total weight of said blend.

24. The process according to claim 22, wherein said sugars are added from 25% to 75% of the total weight of said blend.

25. The process according to claim 20, wherein talc is added from 0.5% to 5% of the total weight of said blend.

26. The process according to claim 20, wherein said sodium phenytoin is added from 35% to 55% of the total weight of said blend.

27. A process for the dry granulation and manufacture of a pharmaceutical formulation, the method comprising the steps of:
adding sodium phenytoin to a vessel of a blender;
adding an excipient to said vessel, wherein said excipient is selected from the group consisting of at least one of stearic acid, magnesium stearate, microcrystalline cellulose, sorbitol, mannitol, confectioner's sugar, compressible sugar, glucose, lactose monohydrate, and talc;
blending said sodium phenytoin and said excipient to form a first blend;
compacting said first blend with sufficient force between at least two rollers to cause a portion of said sodium phenytoin to fracture and form a compact, wherein said rollers apply a force of between 1 and 20 kilo-Newtons to said first blend, said rollers rotate at a speed of between 1 and 20 revolutions per minute, and wherein the outer edge of said rollers are positioned between 1 mm and 5 mm apart at their closest point;
milling said compact to form a granulation; and
blending said granulation to form a second blend.

28. The process according to claim 27, wherein said sodium phenytoin is added to said vessel in an amount of 25% to 75% of the total weight of said granulation.

29. The process according to claim 27, wherein said excipient comprises magnesium stearate, confectioner's sugar, compressible sugar, lactose monohydrate, and talc.

30. The process according to claim 29, wherein said magnesium stearate is added to 1% to 5% of the total weight of said granulation.

31. The process according to claim 29, wherein said sugars are added to 25% to 75% of the total weight of said granulation.

32. The process according to claim 29, wherein said talc is added to 0.5% to 5% of the total weight of said granulation.

33. The process according to claim 27, wherein said sodium phenytoin is added to 35% to 55% of the total weight of said granulation.

34. The process according to claim 27, wherein the step of compacting comprises compacting said sodium phenytoin and said at least one excipient with a force of about 2.5 kN between said rollers, wherein said rollers are rotated at a speed of 10 rpm, and wherein the outer edge of said rollers are positioned 3 mm apart.
